SEQUENCE LISTING (5 sequences)

SEQ ID NO: 1 - protein, 155 aa, Homo sapiens [sequence omitted]

SEQ ID NO: 2 - protein, 196 aa, Homo sapiens
MGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 3 - protein, 201 aa, Homo sapiens
LPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 4 - protein, 210 aa, Homo sapiens
MGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 5 - protein, 288 aa, Homo sapiens
MVGVGGGFVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

(19) United States
(12) Patent Application Publication
(10) Pub. No.: US 2024/0116996 A1
(43) Pub. Date: Apr. 11, 2024

(54) METHODS AND COMPOSITIONS FOR TREATING A CORONAVIRUS INFECTION

(71) Applicant: Recovery Therapeutics, Inc., Needham, MA (US)

(72) Inventor: Seth P. FINKLESTEIN, Needham, MA (US)

(21) Appl. No.: 18/286,316

(22) PCT Filed: Apr. 5, 2022
(86) PCT No.: PCT/US2022/023478

§ 371 Date: Oct. 10, 2023

Related U.S. Application Data
(60) Provisional application No. 63/171,823, filed on Apr. 7, 2021.

(51) Int. Cl. C07K 14/50; A61K 31/706; A61K 31/727; A61P 31/14

(52) U.S. Cl. CPC C07K 14/503; A61K 31/706; A61K 31/727; A61P 31/14

(57) ABSTRACT

The disclosure provides methods of treating an infection by a coronavirus with a fibroblast growth factor (“FGF”). Also provided herein are FGFs for treating an infection by a coronavirus and their use in the manufacture of a medicament for treating an infection by a coronavirus.

### STATEMENT REGARDING FEDERALLY SPONSORED RESEARCH

This invention was made with government support under grant number NS095381 from the National Institutes of Health. The government has certain rights to the invention.

### BACKGROUND OF THE DISCLOSURE

Coronaviruses are enveloped viruses having a capsid having a helical symmetry. They have a positive-sense single-stranded RNA genome and can infect the cells of birds and mammals. In particular, this very large family of viruses is known to include human pathogens that cause, among other conditions and symptoms, colds (e.g., the HCoV and OC43 viruses), bronchiolitis (e.g., the NL63 virus), and even severe pneumonia (e.g., SARS-CoV-2).

There is a continuous need for novel therapies that are effective in treating infections or diseases caused by coronaviruses.

### SUMMARY OF THE DISCLOSURE

The disclosure features the use of fibroblast growth factors (FGFs, e.g., FGF-2) for the treatment of coronavirus infections, such as infections by human coronavirus 0043 (HCoV-0043), human coronavirus HKU1 (HCoV-HKU1), human coronavirus 229E (HCoV-229E), human coronavirus NL63 (HCoV-NL63), Middle East respiratory syndrome coronavirus (MERS-CoV), severe acute respiratory syndrome coronavirus (SARS-CoV), and severe acute respiratory syndrome coronavirus 2 (SARS-CoV-2).

In a first aspect, the present disclosure provides a method of treating an infection by a coronavirus in a subject. The method includes administering to the subject a therapeutically effective amount of a fibroblast growth factor (FGF).

In some embodiments, the FGF is (i) a wild-type FGF (e.g., a wild-type FGF-2, such as a protein represented by SEQ ID NO. 1, 2, 3, 4, or 5); (ii) an FGF mutein (e.g., an FGF-2 mutein, such as a mutein of a protein represented by SEQ ID NO. 1, 2, 3, 4, or 5); (iii) a truncated wild-type FGF (e.g., a truncated FGF-2, in which the FGF-2 may be a protein represented by SEQ ID NO. 1, 2, 3, 4, or 5); or (iv) a truncated FGF mutein (e.g., a truncated FGF-2 mutein, in which the FGF-2 mutein may be a protein represented by SEQ ID NO. 1, 2, 3, 4, or 5), or a chemically modified version thereof.

In some embodiments, the coronavirus is HCoV-0043.

In some embodiments, the coronavirus is HCoV-HKU1.

In some embodiments, the coronavirus is HCoV-229E.

In some embodiments, the coronavirus is HCoV-NL63.

In some embodiments, the coronavirus is MERS-CoV.

In some embodiments, the coronavirus is SARS-CoV.

In some embodiments, the coronavirus is SARS-CoV-2.

In some embodiments, the subject is exhibiting one or more symptoms selected from fatigue; muscle or body aches; congestion; nausea; vomiting; diarrhea; rhinorrhea; coughing; sneezing; lung ground-glass opacity; hemoptysis; nasal congestion; fever; sputum production; sore throat; chills; postnasal discharge; tonsillar hypertrophy; shortness of breath; headache; pneumonia; bronchiolitis; acute respiratory distress syndrome; respiratory failure; respiratory shock; septic shock; cytokine storm; organ failure; hypoxemia; acute cardiac injury; acute lung injury; inflammation of cells in the large away and parenchyma; intra-alveolar edema; lymphopenia; perivascular cuffing; RNAemia; thickening of the interstitial membrane; hemorrhage or clotting, e.g., one or more of venous thromboembolism (e.g., deep vein thrombosis or pulmonary embolism), arterial thrombosis (e.g., stroke or limb ischemia), microvascular thrombosis, and bleeding; and a neurological condition, e.g., one or more of a smell and taste disorder (e.g., anosmia or ageusia), encephalopathy, stroke, venous sinus thrombosis, a neuromuscular disease (e.g., Guillain-Barre syndrome), meningoencephalitis, encephalomyelitis, multisystem inflammatory syndrome, myoclonus, and posterior reversible encephalopathy syndrome (“PRES”). In some embodiments, in which the coronavirus is SARS-CoV-2, the subject is exhibiting post-COVID syndrome.

In some embodiments, the risk of death of the subject is reduced.

In some embodiments, the risk or duration of hospitalization of the subject is reduced.

In some embodiments, the risk of developing one or more symptoms selected from fatigue; muscle or body aches; congestion; nausea; vomiting; diarrhea; rhinorrhea; coughing; sneezing; lung ground-glass opacity; hemoptysis; nasal congestion; fever; sputum production; sore throat; chills; postnasal discharge; tonsillar hypertrophy; shortness of breath; headache; pneumonia; bronchiolitis; acute respiratory distress syndrome; respiratory failure; respiratory shock; septic shock; cytokine storm; organ failure; hypoxemia; acute cardiac injury; acute lung injury; inflammation of cells in the large away and parenchyma; intra-alveolar edema; lymphopenia; perivascular cuffing; RNAemia; thickening of the interstitial membrane; hemorrhage or clotting, e.g., one or more of venous thromboembolism (e.g., deep vein thrombosis or pulmonary embolism), arterial thrombosis (e.g., stroke or limb ischemia), microvascular thrombosis, and bleeding; and a neurological condition, e.g., one or more of a smell and taste disorder (e.g., anosmia or ageusia), encephalopathy, stroke, venous sinus thrombosis, a neuromuscular disease (e.g., Guillain-Barre syndrome), meningoencephalitis, encephalomyelitis, multisystem inflammatory syndrome, myoclonus, and posterior reversible encephalopathy syndrome (“PRES”) is reduced. In some embodiments wherein the coronavirus is SARS-CoV-2, the risk of developing post-COVID syndrome is reduced.

In some embodiments, the risk or duration of need for non-invasive mechanical ventilation is reduced. In some embodiments, the subject is being subjected to non-invasive mechanical ventilation, and the duration of need for non-invasive mechanical ventilation of the subject is reduced.

In some embodiments, the risk or duration of need for extracorporeal membrane oxygenation is reduced.

In some embodiments, the risk or duration of need for invasive mechanical ventilation (e.g., respiratory intubation or high-flow oxygen therapy) is reduced. In some embodiments, the subject is being subjected to invasive mechanical ventilation, and the duration of need for invasive mechanical ventilation is reduced.

In some embodiments, the FGF is administered parenterally (e.g., intravenously, subcutaneously, or intramuscularly).

In some embodiments, the FGF is administered intranasally.

In some embodiments, the FGF is administered via inhalation.

In some embodiments, the subject is being subjected to invasive mechanical ventilation, and the FGF is administered via an endotracheal tube.

In some embodiments, said administering of the FGF includes administering a vector (e.g., a viral vector or a non-viral vector such as a plasmid or a mesenchymal stem cell) comprising a recombinant nucleic acid encoding the FGF.

In some embodiments, said administering of the FGF includes administering an mRNA encoding the FGF.

In some embodiments, the subject has at least one pre-existing condition that increases the risk of one or more of pneumonia, acute respiratory distress syndrome, respiratory failure, septic shock, organ failure, cytokine storm, encephalopathy, stroke, hemorrhage, and death. Pre-existing conditions include, but are not limited to, cardiovascular disease, chronic respiratory disease, diabetes, hypertension, immune deficiency, and obesity.

In some embodiments, the FGF (e.g., FGF-2) is administered in combination with a second therapeutic agent, such as an antiviral agent (e.g., remdesivir), an anti-inflammatory agent (e.g., a steroid or a non-steroidal anti-inflammatory drug), a glycosaminoglycan (e.g., a heparin, a heparinoid, a fractionated low molecular weight heparin, or a heparin salt), or any combination thereof. In some embodiments, the FGF (e.g., FGF-2) and the second therapeutic agent (e.g., an antiviral agent, an anti-inflammatory agent, and/or a glycosaminoglycan) are administered concurrently, and, in further embodiments, are formulated as a single formulation. In some embodiments, the FGF (e.g., FGF-2) and the second therapeutic agent are administered sequentially (e.g., within 1 hour, 1 hour apart, 2 hours apart, 3 hours apart, or 6 hours apart).

In some embodiments, the FGF is FGF-2 and is administered in combination with a glycosaminoglycan (e.g., a heparin, a heparinoid, a fractionated low molecular weight heparin, or a heparin salt) in a single formulation, wherein the glycosaminoglycan is provided at a non-anticoagulant dose. For example, the FGF-2 and glycosaminoglycan may be provided at a 1:1, 2:1, 3:1, 4:1 or 5:1 molar ratio.

In a second aspect, the present disclosure provides an FGF (e.g., FGF-2) for use in a method of treating an infection by a coronavirus in a subject. The method may be any one of the methods described herein.

In a third aspect, the present disclosure features the use of an FGF (e.g., FGF-2) in the manufacture of a medicament for use in a method of treating an infection by a coronavirus in a subject. The medicament is suitable for use in any of the methods described herein.

### BRIEF DESCRIPTION OF THE DRAWINGS

FIG. 1 is a graph showing the reduction of viral cytopathic effect (CPE) in HAP1 cells infected with Human Coronavirus Strain 229E (HCoV-229E) as a function of remdesivir concentration (closed diamonds) and cell survival in the absence of HCoV-229E as a function of remdesivir concentration (open squares).

FIG. 2 is a graph showing reduction of viral cytopathic effect (CPE) in HAP1 cells infected with HCoV-229E as a function of FGF-2 concentration (closed diamonds) and cell survival in the absence of HCoV-229E as a function of FGF-2 concentration (open squares).

FIG. 3 is a graph showing the lung viral load of male Golden Syrian hamsters infected with SARS-CoV-2. The hamsters received intravenous administration of vehicle (normal saline with 100 μg/mL BSA), 20 μg FGF-2, or 50 μg FGF-2.

### REFERENCE TO SEQUENCE LISTING

The amino acid sequences described herein are shown using standard letter abbreviations for amino acids, as defined in 37 C.F.R. § 1.822. A computer readable text file, entitled “51029-010W02 Sequence Listing 04.05.22_ST25.txt” created on or about Apr. 5, 2022, with a file size of 9559 bytes, contains the sequence listing for this application and is hereby incorporated by reference in its entirety.

SEQ ID NO:1 is the amino acid sequence of a human FGF-2 low molecular weight isoform (18 kDa): MAAGSITTLP ALPEDGGSGA FPPGHFKDPK RLYCKNGGFF LRIHPDGRVD GVREKSDPHI KLQLQAEERG VVSIKGVCAN RYLAMKEDGR LLASKCVTDE CFFFERLESN NYNTYRSRKY TSWYVALKRT GQYKLGSKTG PGQKAILFLP MSAKS.

SEQ ID NO: 2 is the amino acid sequence of a human FGF-2 high molecular weight isoform (22 kDa): MGGRGRGRAP ERVGGRGRGR GTAAPRAAPA ARGSRPGPAG TMAAGSITTL PALPEDGGSG AFPPGHFKDP KRLYCKNGGF FLRIHPDGRV DGVREKSDPH IKLQLQAEER GVVSIKGVCA NRYLAMKEDG RLLASKCVTD ECFFFERLES NNYNTYRSRK YTSWYVALKR TGQYKLGSKT GPGQKAILFL PMSAKS.

SEQ ID NO: 3 is the amino acid sequence of a human FGF-2 high molecular weight isoform (22.5 kDa): LPGGRLGGRG RGRAPERVGG RGRGRGTAAP RAAPAARGSR PGPAGTMAAG SITTLPALPE DGGSGAFPPG HFKDPKRLYC KNGGFFLRIH PDGRVDGVRE KSDPHIKLQL QAEERGVVSI KGVCANRYLA MKEDGRLLAS KCVTDECFFF ERLESNNYNT YRSRKYTSWY VALKRTGQYK LGSKTGPGQK AILFLPMSAK S.

SEQ ID NO: 4 is the amino acid sequence of a human FGF-2 high molecular weight isoform (24 kDa): MGDRGRGRAL PGGRLGGRGR GRAPERVGGR GRGRGTAAPR AAPAARGSRP GPAGTMAAGS ITTLPALPED GGSGAFPPGH FKDPKRLYCK NGGFFLRIHP DGRVDGVREK SDPHIKLQLQ AEERGVVSIK GVCANRYLAM KEDGRLLASK CVTDECFFFE RLESNNYNTY RSRKYTSWYV ALKRTGQYKL GSKTGPGQKA ILFLPMSAKS.

SEQ ID NO: 5 is the amino acid sequence of a human FGF-2 high molecular weight isoform (34 kDa): MVGVGGGFVE DVTPRPGGCQ ISGRGARGCN GIPGAAAWEA ALPRRRPRRH PSVNPRSRAA GSPRTRGRRT EERPSGSRLG DRGRGRALPG GRLGGRGRGR APERVGGRGR GRGTAAPRAA PAARGSRPGP AGTMAAGSIT TLPALPEDGG SGAFPPGHFK DPKRLYCKNG GFFLRIHPDG RVDGVREKSD PHIKLQLQAE ERGVVSIKGV CANRYLAMKEDGRLLASKCV TDECFFFERL ESNNYNTYRS RKYTSWYVAL KRTGQYKLGS KTGPGQKAIL FLPMSAKS.

### Definitions

To facilitate the understanding of this disclosure, a number of terms are defined below. Terms defined herein have meanings as commonly understood by a person of ordinary skill in the areas relevant to the disclosure. Terms such as “a”, “an,” and “the” are not intended to refer to only a singular entity, but include the general class of which a specific example may be used for illustration. The terminology herein is used to describe specific embodiments of the disclosure, but their usage does not limit the disclosure, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the terms “fibroblast growth factor” and “FGF,” as used herein, include (i) wild-type FGFs (including all isoforms/splice variants); (ii) FGF muteins; (iii) truncated wild-type FGFs or FGF muteins; and (iv) chemically modified wild-type FGFs, FGF muteins, truncated wild-type FGFs, and truncated FGF muteins. An FGF mutein is an FGF in which one or more amino acids of the wild-type FGF are replaced with different amino acids (natural amino acids and/or synthetic amino acids). An FGF mutein may be a synthetic protein or a mutant protein encoded by a mutated nucleic acid. In some embodiments, an FGF mutein has at least 30% (e.g., at least 35%, at least 40%, at least 45%, at least 50%, at least 55%, at least 60%, at least 65%, at least 70%, at least 75%, at least 80%, at least 85%, at least 90%, or at least 95%) sequence identity to the amino acid sequence of the wild-type FGF. In some embodiments, a truncated wild-type FGF or FGF mutein is a wild-type FGF or FGF mutein shortened at the N-terminus and/or C-terminus by one or more amino-acid residues (e.g., by 1-25, 1-20, 1-15, 1-10, or 1-5 amino acids). Chemical modifications to FGFs include, but are not limited to, N-formylation, oxidation, deamidation, isomerization, decarboxylation, phosphorylation, glycosylation, lipidation, gluconoylation, phosphoglycosylation, hydroxylation, sulphation, methylation, and carbamylation. FGF also includes forms isolated from a particular species, e.g., human, chemically synthesized, or recombinant. FGF further includes post translational modified versions from a host cell (e.g., human or CHO) or the unmodified version. In some embodiments, the FGF is an FGF mutein, a truncated wild-type FGF, a truncated FGF mutein, or a chemically modified version thereof having at least 30% (e.g., at least 35%, at least 40%, at least 45%, at least 50%, at least 55%, at least 60%, at least 65%, at least 70%, at least 75%, at least 80%, at least 85%, at least 90%, or 95% or more) of the biological activity (such as antiviral activity as measured by a viral infection assay of a wild-type FGF, e.g., as described in Example 1.

As used herein, the term “pharmaceutical composition” refers to one or more active compounds, formulated together with one or more pharmaceutically acceptable excipients. In some embodiments, an active compound (e.g., an FGF such as FGF-2) is present in a unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous, or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The term “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes, emollients, emulsifiers, diluents, film formers or coatings, flavors, fragrances, glidants, lubricants, preservatives, printing inks, sorbents, suspending or dispersing agents, sweeteners, or waters of hydration. Excipients include, but are not limited to: butylated optionally substituted hydroxytoluene (e.g., BHT), calcium carbonate, calcium phosphate dibasic, calcium stearate, croscarmellose, crosslinked polyvinyl pyrrolidone, citric acid, crospovidone, cysteine, ethylcellulose, gelatin, optionally substituted hydroxypropyl cellulose, optionally substituted hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch, stearic acid, stearic acid, sucrose, talc, titanium dioxide, vitamin A, vitamin E, vitamin C, and xylitol. Those of ordinary skill in the art are familiar with a variety of agents and materials useful as excipients.

As used herein, “reducing the risk of death” refers to reducing the frequency of deaths among subjects treated according to any of the methods of the disclosure. The reduction is in comparison to control subjects of the same age, sex, and/or condition (e.g., comorbidities) that are untreated. In some embodiments, the frequency of death is reduced by at least 10% (e.g., at least 20%, at least 30%, at least 40%, at least 50%, at least 60%, at least 70%, at least 80%, at least 90%, or 99% or more) relative to the frequency of deaths observed for the control subjects.

As used herein, “reducing the risk of hospitalization” refers to reducing the frequency of hospitalization in subjects treated according to any of the methods of the disclosure, and “reducing the duration of hospitalization” refers to reducing the duration of hospitalization in subjects treated according to any of the methods of the disclosure. The reduction is in comparison to control subjects of the same age, sex, and/or condition (e.g., comorbidities) that are untreated. In some embodiments, the frequency or duration of hospitalization is reduced by at least 10% (e.g., at least 20%, at least 30%, at least 40%, at least 50%, at least 60%, at least 70%, at least 80%, at least 90%, or 99% or more) relative to the frequency or duration of hospitalization observed for the control subjects.

As used herein, “reducing the risk of need for extracorporeal membrane oxygenation” refers to reducing the frequency of use of extracorporeal membrane oxygenation in subjects treated according to any of the methods of the disclosure, and “reducing the duration of need for extracorporeal membrane oxygenation” refers to reducing the duration of continuous use of extracorporeal membrane oxygenation in subjects treated according to any of the methods of the disclosure. The reduction is in comparison to control subjects of the same age, sex, and/or condition (e.g., comorbidities) that are untreated. In some embodiments, the frequency or duration of continuous use of extracorporeal membrane oxygenation is reduced by at least 10% (e.g., at least 20%, at least 30%, at least 40%, at least 50%, at least 60%, at least 70%, at least 80%, at least 90%, or 99% or more) relative to the frequency or duration of continuous use of extracorporeal membrane oxygenation observed for the control subjects.

As used herein, “reducing the risk of need for invasive mechanical ventilation” in a subject refers to reducing the frequency of use of invasive mechanical ventilation (e.g., respiratory intubation or high-flow oxygen therapy) in subjects treated according to any of the methods of the disclosure, and “reducing the duration of need for invasive mechanical ventilation” in a subject refers to reducing the duration of continuous use of invasive mechanical ventilation (e.g., respiratory intubation or high-flow oxygen therapy) in subjects treated according to any of the methods of the disclosure. The reduction is in comparison to control subjects of the same age, sex, and/or condition (e.g., comorbidities) that are untreated. In some embodiments, the frequency or duration of continuous use of invasive mechanical ventilation (e.g., respiratory intubation or high-flow oxygen therapy) is reduced by at least 10% (e.g., at least 20%, at least 30%, at least 40%, at least 50%, at least 60%, at least 70%, at least 80%, at least 90%, or 99% or more) relative to the frequency or duration of continued use of invasive mechanical ventilation (e.g., respiratory intubation or high-flow oxygen therapy) observed for the control subjects.

As used herein, “reducing the risk of need for non-invasive mechanical ventilation” in a subject refers to reducing the frequency of use of non-invasive mechanical ventilation in subjects treated according to any of the methods of the disclosure, and “reducing the duration of need for non-invasive mechanical ventilation” in a subject refers to reducing the duration of continuous use of non-invasive mechanical ventilation in subjects treated according to any of the methods of the disclosure. The reduction is in comparison to control subjects of the same age, sex, and/or condition (e.g., comorbidities) that are untreated. In some embodiments, the frequency or duration of continuous use of non-invasive mechanical ventilation is reduced by at least 10% (e.g., at least 20%, at least 30%, at least 40%, at least 50%, at least 60%, at least 70%, at least 80%, at least 90%, or 99% or more) relative to the frequency or duration of continuous use of non-invasive mechanical ventilation observed for the control subjects.

The term “subject,” as used herein, can be a human, non-human primate, or other mammal, such as but not limited to dog, cat, horse, cow, pig, goat, monkey, rat, mouse, hamster, ferret, and sheep. As used herein, the term “therapeutically effective amount” refers to an amount of an active compound (e.g., an FGF such as FGF-2) required to treat, ameliorate the symptoms of, or inhibit the progression of a coronavirus infection. In some embodiments, the effective amount of the active compound (e.g., an FGF such as FGF-2) refers to an amount of an active compound that reduces one or more of (1) risk of death, (2) risk or duration of hospitalization, (3) risk or duration of need for extracorporeal membrane oxygenation, (4) risk or duration of need for invasive mechanical ventilation, and (5) risk or duration of need for non-invasive mechanical ventilation. The effective amount of the active compound (e.g., an FGF such as FGF-2) varies depending upon the manner of administration, the age, body weight, and/or general health of the subject. Ultimately, the attending physician will decide the therapeutically effective amount of the active compound (e.g., an FGF such as FGF-2) and dosing regimen.

As used herein, and as well understood in the art, “to treat” a condition or “treatment” of various diseases and disorders is an approach for obtaining beneficial or desired results, such as clinical results. Beneficial or desired results can include, but are not limited to, alleviation of one or more symptoms; diminishment of extent of disease; stabilizing (i.e., not worsening) state of disease; delaying, slowing, or inhibiting the progress of the disease; amelioration or palliation of the disease; and remission (whether partial or total), whether detectable or undetectable. “Palliating” a disease means that the extent and/or undesirable clinical manifestations of the disease are lessened and/or time course of the progression is slowed or lengthened, as compared to the extent or time course in the absence of treatment. In some embodiments, the treatment reduces at least one symptom (e.g., any one of the symptoms described herein) of a coronavirus infection and/or delays onset of at least one symptom of the coronavirus infection in a subject as compared to what is observed in control subjects, e.g., those of the same age, sex, and/or condition (e.g., comorbidities), that are untreated. In some embodiments, the treatment reduces one or more of (1) the risk of death, (2) the risk or duration of hospitalization, (3) the risk or duration of need for extracorporeal membrane oxygenation, (4) the risk or duration of need for invasive mechanical ventilation, and (5) the risk or duration of need for non-invasive mechanical ventilation.

### DETAILED DESCRIPTION OF THE DISCLOSURE

The present disclosure generally relates to methods for treating infections by coronaviruses, such as HCoV-0043, HCoV-HKU1, HCoV-229E, HCoV-NL63, MERS-CoV, SARS-CoV, and SARS-CoV-2. The methods include administering an FGF (e.g., FGF-2) to a subject suffering from a coronavirus infection.

### Coronavirus Infections

Coronaviruses are a group of related RNA viruses that cause mild to lethal respiratory tract infections in humans and other animals such as hamsters, mice, ferrets, other non-human primates, and birds. Known species of human coronaviruses include four that generally produce mild symptoms, i.e., HCoV-0043, HCoV-HKU1, HCoV-229E, and HCoV-NL63, and three that are known to produce symptoms that are potentially severe, i.e., MERS-CoV, SARS-CoV, and SARS-CoV-2. Symptoms of coronavirus infections include, but are not limited to, fatigue, muscle or body aches, congestion, nausea, vomiting, diarrhea, rhinorrhea, coughing, sneezing, nasal congestion, fever, sputum production, sore throat, chills, postnasal discharge, tonsillar hypertrophy, shortness of breath, headache, pneumonia, and bronchiolitis.

Severe symptoms of coronavirus infections (e.g., those associated with SARS-CoV-2) include, but are not limited to lung ground-glass opacity; hemoptysis; acute respiratory distress syndrome; respiratory failure; respiratory shock; septic shock; cytokine storm; organ failure; hypoxemia; acute cardiac injury; acute lung injury; inflammation of cells in the large away and parenchyma; lymphopenia; perivascular cuffing; RNAemia; thickening of the interstitial membrane, hematological complications such as thromboembolism (e.g., deep vein thrombosis or pulmonary embolism), arterial thrombosis (e.g., stroke or limb ischemia), microvascular thrombosis, and bleeding; and neurological conditions such as smell and taste disorders (e.g., anosmia or ageusia), encephalopathy, stroke, venous sinus thrombosis, neuromuscular diseases (e.g., Guillain-Barre syndrome), meningoencephalitis, encephalomyelitis, multisystem inflammatory syndrome, myoclonus, and posterior reversible encephalopathy syndrome (“PRES”). Infection by SARs-CoV-2 is known to cause post-COVID syndrome (see, e.g., Mayo Clinic (2020) “COVID-19 (coronavirus): Long-term effects,” https[[://]]www.mayoclinic.org/diseases-conditions/coronavirus/in-depth/coronavirus-long-term-effects/art-20490351).

### Fibroblast Growth Factors

As set forth above, the present disclosure relates to the use of FGFs in the treatment of an infection by a coronavirus, e.g., HCoV-0C43, HCoV-HKU1, HCoV-229E, HCoV-NL63, MERS-CoV, SARS-CoV, or SARS-CoV-2. There are 23 identified members of the FGF family binding to four identified high-affinity tyrosine kinase receptors and their splice variants. FGFs play important roles in the development, survival, growth, differentiation, proliferation, and repair of cells and tissues.

In general, the FGF may be a wild-type FGF; an FGF mutein; a truncated wild-type FGF or FGF mutein; or a chemically modified wild-type FGF or FGF mutein (full length or truncated). In some embodiments, the FGF is FGF-2, e.g., a protein represented by SEQ ID NO.: 1, 2, 3, 4, or 5. The FGF-2 may be an isolated, naturally occurring FGF-2 or FGF-2 that is recombinantly expressed. Human FGF-2 is commercially available, and recombinant FGF-2 can be prepared according to methods known in the art. See, e.g., U.S. Pat. No. 5,155,214, the contents of which is incorporated herein by reference in its entirety.

In some embodiments, the FGF (e.g., FGF-2) is expressed in vivo. For example, a subject may be administered a vector comprising a recombinant nucleic acid or an mRNA encoding the FGF (e.g., FGF-2). The vector may be a viral vector (see, e.g., Madry et al., Am J Sports Med. 2013; 41(1): 194-202) or a non-viral vector, such as a plasmid (see, e.g., Ferraro et al., Gene Therapy. 2010; 17: 763-769) or a mesenchymal stem cell (see, e.g., Zhao et al., Eur Rev Med Pharmacol Sci. 2015; 19(5): 857-65).

### Methods of Treatment

The dosage of the FGF (e.g., FGF-2) depends on factors including the disease to be treated and physical characteristics, e.g., age, weight, and general health, of the subject. Typically, the amount of FGF (e.g., FGF-2) contained within a single dose may be an amount that effectively treats the disease without inducing significant toxicity.

A dosage of an FGF (e.g., FGF-2) ranges from about 0.01 to about 500 μg/kg/day (e.g., about 1 μg/kg/day to about 450 μg/kg/day, about 5 μg/kg/day to about 400 μg/kg/day, about 10 μg/kg/day to about 350 μg/kg/day, about 20 μg/kg/day to about 300 μg/kg/day, about 30 μg/kg/day to about 250 μg/kg/day, about 40 μg/kg/day to about 200 μg/kg/day, about 50 μg/kg/day to about 175 μg/kg/day, or about 60 μg/kg/day to about 150 μg/kg/day). The dosage may be adapted by the clinician in accordance with conventional factors such as the extent of the disease and different parameters of the subject. Typically, an FGF (e.g., FGF-2) can be administered in an amount from about 0.001 mg to about 500 mg (e.g., about 0.05 mg, about 0.01 mg, about 0.1 mg, about 0.2 mg, about 0.3 mg, about 0.5 mg, about 0.7 mg, about 0.8 mg, about 1 mg, about 2 mg, about 3 mg, about 4 mg, about 5 mg, about 10 mg, about 15 mg, about 20 mg, about 30 mg, about 50 mg, about 100 mg, about 250 mg, or about 500 mg), e.g., within or over 24 hours.

In some embodiments, the treatment of a coronavirus infection with an FGF (e.g., FGF-2) reduces the viral load of the infection (e.g., by at least 5%, by at least 10%, by at least 15%, but at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 95% or more) relative to either pre-treatment levels in the same subject, or relative to subjects of the same age, sex, and/or condition (e.g., comorbidities) having the same type of infection who have not received treatment, e.g., as measured by CCID50, q PCR, and/or inhibition of viral cytopathic effect using a cytopathic effect inhibition assay.

In some embodiments, the treatment of a coronavirus infection with an FGF (e.g., FGF-2) reduces the length of time associated with resolution of the infection by 20% or more (e.g., 30% or more, 35% or more, 40% or more, 45% or more, 50% or more, 55% or more, 60% or more, 65% or more, 70% or more, 80% or more, 85% or more, or 90% or more), as compared to an infection of the same type in subjects of the same age, sex, and/or condition (e.g., comorbidities) who have not received treatment.

In some embodiments, the treatment of a coronavirus infection with an FGF (e.g., FGF-2) reduces the risk of death (e.g., by at least 10%, by at least 20%, by at least 30%, but at least 40%, by at least 50%, by at least 60%, by at least 70%, by at least 80%, by at least 90%, or by 99% or more) in subjects treated according to any of the methods of the disclosure compared to control subjects of the same age, sex, and/or condition (e.g., comorbidities) that are untreated.

In some embodiments, the treatment of a coronavirus infection with an FGF (e.g., FGF-2) reduces the risk or duration of hospitalization (e.g., by at least 10%, by at least 20%, by at least 30%, but at least 40%, by at least 50%, by at least 60%, by at least 70%, by at least 80%, by at least 90%, or by 99% or more) in subjects treated according to any of the methods of the disclosure as compared to control subjects of the same age, sex, and/or condition (e.g., comorbidities) that are untreated.

In some embodiments, the treatment of a coronavirus infection with an FGF (e.g., FGF-2) reduces the risk or duration of need for extracorporeal membrane oxygenation (e.g., by at least 10%, by at least 20%, by at least 30%, but at least 40%, by at least 50%, by at least 60%, by at least 70%, by at least 80%, by at least 90%, or by 99% or more) in subjects treated according to any of the methods of the disclosure as compared to control subjects of the same age, sex, and/or condition (e.g., comorbidities) that are untreated.

In some embodiments, the treatment of a coronavirus infection with an FGF (e.g., FGF-2) reduces the risk or duration of need for invasive mechanical ventilation (e.g., by at least 10%, by at least 20%, by at least 30%, but at least 40%, by at least 50%, by at least 60%, by at least 70%, by at least 80%, by at least 90%, or by 99% or more) in subjects treated according to the any of the methods of the disclosure as compared to control subjects of the same age, sex, and/or condition (e.g., comorbidities) that are untreated.

In some embodiments, the treatment of a coronavirus infection with an FGF (e.g., FGF-2) reduces the risk or duration of need for non-invasive mechanical ventilation (e.g., by at least 10%, by at least 20%, by at least 30%, but at least 40%, by at least 50%, by at least 60%, by at least 70%, by at least 80%, by at least 90%, or by 99% or more) in subjects treated according to any of the methods of the disclosure as compared to control subjects of the same age, sex, and/or condition (e.g., comorbidities) that are untreated.

In some embodiments, the treatment of a coronavirus infection with an FGF (e.g., FGF-2) reduces the risk of developing one or more symptoms disclosed herein is reduced (e.g., e.g., by at least 10%, by at least 20%, by at least 30%, but at least 40%, by at least 50%, by at least 60%, by at least 70%, by at least 80%, by at least 90%, or by 99% or more) in subjects treated according to any of the methods of the disclosure as compared to control subjects of the same age, sex, and/or condition (e.g., comorbidities) that are untreated.

### Combination Therapy

In some embodiments, the FGF (e.g., FGF-2) is formulated with or administered concurrently with, prior to, or subsequent to, one or more additional therapeutic agents. The particular combination of FGF (e.g., FGF-2) and the one or more additional therapeutic agents will take into account of the compatibility of the one or more additional therapeutic agents and the FGF (e.g., FGF-2) and the desired therapeutic effect to be achieved. For example, the one or more additional therapeutic agents may be effective in treating a coronavirus infection and/or symptoms thereof.

When a subject is treated with a combination of FGF (e.g., FGF-2) and one or more additional therapeutic agents, the FGF (e.g., FGF-2) and the one of more additional therapeutic agents may be administered sequentially (e.g., 1 day apart, 2 days apart, 3 days apart, 1 week apart, 1 month apart, 6 months apart, or more) or substantially simultaneously (e.g., within 1 day). The FGF (e.g., FGF-2) and the one or more therapeutic agents may be formulated in a single pharmaceutical composition or may be administered as separate pharmaceutical compositions, e.g., by the same route of administration or by different routes of administration, at the same frequency or at different frequencies.

In some embodiments, the FGF (e.g., FGF-2) is administered in combination with an antiviral agent (e.g., remdesivir), an anti-inflammatory agent (e.g., a steroid or a non-steroidal anti-inflammatory agent) and/or a glycosaminoglycan (e.g., a heparin, a heparinoid, a fractionated low molecular weight heparin, or a heparin salt). In some embodiments, the FGF (e.g., FGF-2) and the second therapeutic agent are administered sequentially (e.g., within 1 hour, 1 hour apart, 2 hours apart, 3 hours apart, or 6 hours apart).

### Pharmaceutical Compositions

Pharmaceutical compositions of the disclosure typically contain a therapeutically effective amount of an active agent (e.g., an FGF, such as FGF-2 and/or a second therapeutic agent) and a pharmaceutically acceptable excipient. The pharmaceutical compositions can be formulated by methods known to those skilled in the art.

Exemplary routes of administration of the pharmaceutical compositions include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration.

The FGF (e.g., FGF-2) may be formulated in manners that preserve the biological activity of the FGF (e.g., FGF-2) in aqueous solutions. For example, the FGF (e.g., FGF-2) may be conjugated to heparin/heparin-like molecules or inorganic materials (e.g., calcium phosphate granules); formulated or co-administered (concurrently or sequentially, e.g., via the same route or different routes of administration) with heparin/heparin-like molecules; or stabilized by using a physical barrier (e.g., polymer matrices, polymer microspheres, hydrogels, or other composite scaffolds). In some embodiments, the FGF-2 is PEGylated (see, e.g. Zhao et al., Biotechnol. J. 2020; 15 (2): e1900203); enclosed by a liposome (see, e.g., Rusnati et al., Mol. Biol. Cell. 1996; 7: 369-381) or a microsphere (a microsphere in a water/oil/water emulsion or an alginate:collagen microsphere (see, e.g., Ali et al., Biol. Open. 2018; 7; and Lotz et al., PLoS ONE. 2013; 8: e56289), or adsorbed onto a surface of a nanoparticle (e.g., a bioactive glass nanoparticle formed from polylactic acid; see, e.g., Yoon et al., RSC Adv. 2017; 7: 16453-16459). Methods of stabilizing FGF-2 in aqueous solutions are known in the art.

Formulations for Oral Administration

The pharmaceutical compositions of the disclosure include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Pharmaceutical compositions for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compositions of the present disclosure can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

In some embodiments, a pharmaceutical composition formulated for administration via an endotracheal tube, e.g., as a solution, a suspension, a powder, or granules of an active compound. In some embodiments, a pharmaceutical composition is formulated for administration via an inhaler, e.g., as a powder or as granules of an active compound.

Formulations for Parenteral Administration

The pharmaceutical compositions of the disclosure can be administered in a pharmaceutically acceptable parenteral (e.g., intravenous, intramuscular, subcutaneous or the like) formulation as described herein. The pharmaceutical composition may also be administered parenterally in dosage forms or formulations containing conventional, non-toxic pharmaceutically acceptable carriers and adjuvants. In particular, formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. For example, to prepare such a composition, the active compound may be dissolved or suspended in a parenterally acceptable liquid vehicle. Among acceptable vehicles and solvents that may be employed are water; water adjusted to a suitable pH by addition of an appropriate amount of hydrochloric acid, sodium hydroxide, or a suitable buffer; 1,3-butanediol; Ringer's solution; and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP 43 NF 38), herein incorporated by reference in its entirety.

The parenteral formulation can be any of the five general types of preparations identified by the USP-NF as suitable for parenteral administration:
(1) “Drug Injection:” a liquid preparation that is a drug substance, or a solution thereof;
(2) “Drug for Injection:” the drug substance as a dry solid that will be combined with the appropriate sterile vehicle for parenteral administration as a drug injection;
(3) “Drug Injectable Emulsion:” a liquid preparation of the drug substance that is dissolved or dispersed in a suitable emulsion medium;
(4) “Drug Injectable Suspension:” a liquid preparation of the drug substance suspended in a suitable liquid medium; and
(5) “Drug for Injectable Suspension:” the drug substance as a dry solid that will be combined with the appropriate sterile vehicle for parenteral administration as a drug injectable suspension.

Exemplary formulations for parenteral administration include solutions of the compound prepared in water suitably mixed with a surfactant, e.g., hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 23^{rd} Ed., Adejare, Ed., Academic Press (2020) and in The United States Pharmacopeia and National Formulary (USP 43 NF38), published in 2019.

Formulations for parenteral administration may, for example, contain sterile water, saline, polyalkylene glycols (e.g., polyethylene glycol), oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

Formulations for Intranasal Administration, Inhalation, or Administration Via an Endotracheal Tube

The pharmaceutical compositions of the disclosure include those formulated for intranasal administration and/or inhalation (e.g., nasal drops or nasal sprays), or administration via an endotracheal tube. Such formulations may contain, for example, lactose, or may be aqueous solutions containing, for example, polyoxyethylene-9-lauryl ether, glycocholate and deoxycholate, or may be oily solutions for administration in the form of nasal drops a gel, a powder, or as granules.

### EXAMPLES

### Example 1. Activity of Fibroblast Growth Factor 2 (FGF-2) Against Human Coronavirus Strain 229E

A study was performed to evaluate the activity of FGF-2 (SEQ ID NO. 1) against the human coronavirus strain 229E (HCoV-229E) in HAP1 cells using a cytopathic inhibition assay. HAP1 cells are a human cell line that express FGF-2 as well as its major receptor FGFR1.

HAP1 (C859) cells were acquired from Horizon Discovery and propagated as recommended by the supplier. Human coronavirus strain 229E (ATCC VR-740) was purchased from the ATCC and grown as recommended by the ATCC. Recombinant human FGF-2 was purchased from Sigma-Aldrich (catalog #F0291) and was solubilized at 25 mg/mL in 100 mg/mL of bovine serum albumin (BSA). FGF-2 was aliquoted and stored at −20° C. BSA was purchased from Sigma Aldrich (catalog number 711454) and solubilized at 20 mg/mL in DPBS. The BSA was prepared fresh prior to solubilization.

HAP1 cells (passage 8) were added to a 96-well plate at a density of 1×10 4 cells/well in DMEM supplemented with 100 mL 10% fetal bovine serum (FBS) 48 hours prior to the addition of the virus. 24 hours prior to the addition of virus (D-1), FGF-2 was diluted 1:2 in assay media (DMEM+2% FBS) and 6 concentrations were added to the cells in a volume of 100 mL in triplicate. Concentrations ranged from 1 ng/mL to 0.003 ng/mL. On the day of virus addition (DO), the virus was diluted to a titer of 0.4 mL/well in assay medium and added to the experimental wells and the virus control wells in a volume of 100 mL. Although the activity of remdesivir in these cells under these assay conditions was unknown, it was evaluated in parallel as a control inhibitor in the absence of FGF-2. On the day of virus addition and daily for two days thereafter (DO, D1,D2), 10 mL of FGF-2 at each concentration was added to the wells. 10 mL of assay media was added to the remdesivir wells, virus control wells, and cell control wells. Following 4 days in culture at 37° C./5% CO₂, the cells were stained with 2,3-bis(2-methoxy-4-nitro-5-sulfophenyl)-5-[(phenylamino)carbonyl]-2H-tetrazolium hydroxide (XTT) to evaluate cellular viability.

XTT in metabolically active cells is metabolized by the mitochondrial enzyme NADPH oxidase to a soluble formazan product. XTT solution was prepared daily as a stock of 1 mg/mL in DMEM without additives. Phenazine methosulfate (PMS) solution was prepared at 0.15 mg/mL in DPBS and stored in the dark at −20° C. XTT/PMS stock was prepared immediately before use by adding 40 μL of PMS per mL of XTT solution. 50 μL of XTT/PMS was added to each well of the plate, and the plate was incubated for 4 hours at 37° C. The 4-hour incubation has been empirically determined to be within the linear response range for XTT dye reduction with the indicated numbers of cells for each assay. The plates were sealed and inverted several times to mix the soluble formazan product and the plate was read at 450 nm (650 nm reference wavelength) with a Molecular Devices SpectraMax Plus 384 96 well plate format spectrophotometer.

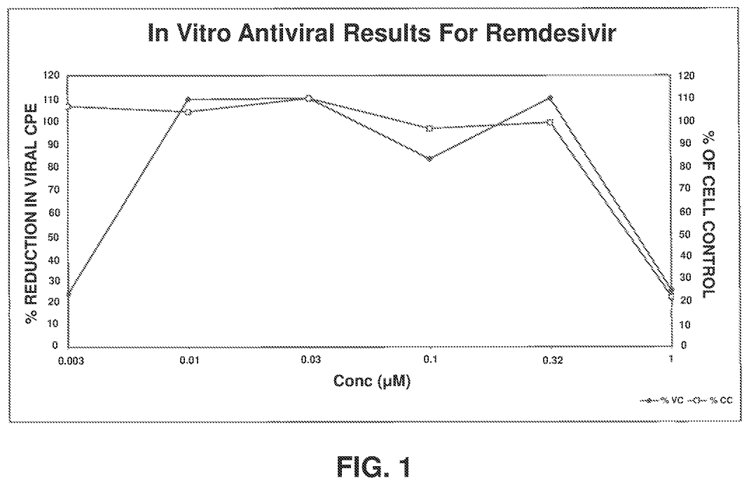

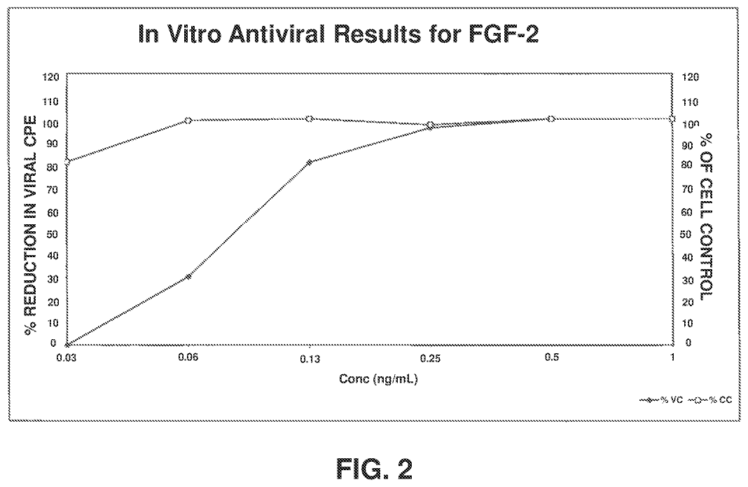

The results of the study are shown in FIGS. 1 (remdesivir) and 2 (FGF-2). Importantly, FIG. 2 shows the dose-dependent enhancement of cell survival without toxicity in the presence of increasing concentrations of FGF-2, with an EC₅₀ value of 0.08 ng/mL.

### Example 2. Intravenous FGF-2 Decreases Lung Viral Load in Hamsters Following COVID-19 Infection

A study was conducted to determine the effects of intravenous FGF-2 on lung viral load in hamsters infected with SARS-CoV-2.

Male Golden Syrian hamsters (6-8 weeks of age) received intranasal administration of SARS-CoV-2 (strain USA-WA1/2020, 6,000 PFU/animal) on Day 0 (DO). On D-1, DO, D1, and D2, the animals received an intravenous injection of either 0 μg (control), 20 μg, or 50 μg of FGF-2 (PeproTech, 100-18B) in normal saline with 100 μg/mL BSA (6 animals/group).

On D6 following virus administration, the animals were euthanized, and the lungs were removed for analysis with a TCID₅₀ viral assay. Viral load was expressed as TCID₅₀/g m tissue.

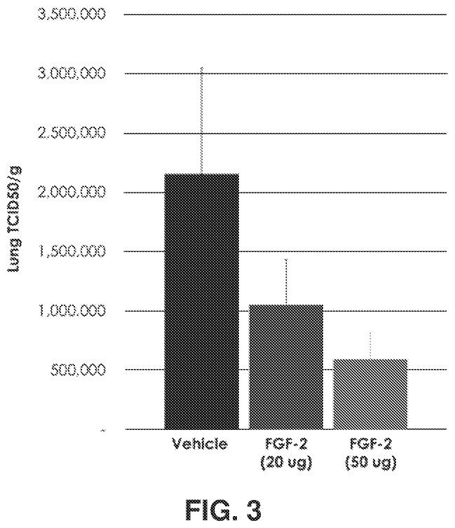

Compared to vehicle-treated animals, treatment with FGF-2 at 20 μg or 50 μg showed 51% or 73% reductions in viral load, respectively (see FIG. 3).

### OTHER EMBODIMENTS

While the disclosure describes specific embodiments of methods, compounds, and uses, it will be understood that further modifications can be made thereto, and this application is intended to cover any variations or adaptations thereof following, in general, the principles of the disclosure including such departures from the disclosure that come within known or customary practice within the art to which the disclosure pertains and may be applied to essential features hereinbefore set forth, and follows in the scope of the claims. Other embodiments are within the claims.

What is claimed is:

1. A method of treating an infection by a coronavirus in a subject, the method comprising administering to the subject a therapeutically effective amount of a fibroblast growth factor (FGF).

2. The method of claim 1, wherein the subject is human.

3. The method of claim 1 or 2, wherein the FGF is FGF-2 or a chemically modified version thereof.

4. The method of claim 3, wherein the FGF is a wild-type FGF-2 or a chemically modified version thereof.

5. The method of claim 3 wherein the FGF is a truncated wild-type FGF-2 or a chemically modified version thereof.

6. The method of claim 4 or 5, wherein the wild-type FGF-2 is a protein represented by SEQ ID NO. 1, 2, 3, 4, or 5.

7. The method of claim 3, wherein the FGF is a protein represented by SEQ ID NO. 1.

8. The method of claim 3, wherein the FGF is an FGF-2 mutein or a chemically modified version thereof.

9. The method of claim 3, wherein the FGF is a truncated FGF-2 mutein or a chemically modified version thereof.

10. The method of claim 8 or 9, wherein the FGF-2 mutein is a mutein of a protein represented by SEQ ID NO. 1, 2, 3, 4, or 5.

11. The method of any one of claims 1-10, wherein the coronavirus is HCoV-0043.

12. The method of any one of claims 1-10, wherein the coronavirus is HCoV-HKU1.

13. The method of any one of claims 1-10, wherein the coronavirus is HCoV-229E.

14. The method of any one of claims 1-10, wherein the coronavirus is HCoV-NL63.

15. The method of any one of claims 1-10, wherein the coronavirus is MERS-CoV.

16. The method of any one of claims 1-10, wherein the coronavirus is SARS-CoV.

17. The method of any one of claims 1-10, wherein the coronavirus is SARS-CoV-2.

18. The method of any one of claims 1-17, wherein the subject is exhibiting one or more symptoms selected from fatigue, muscle or body aches, congestion, nausea, vomiting, diarrhea, rhinorrhea, coughing, sneezing, lung ground-glass opacity, hemoptysis, nasal congestion, fever, sputum production, sore throat, chills, postnasal discharge, tonsillar hypertrophy, shortness of breath, headache, pneumonia, bronchiolitis, acute respiratory distress syndrome, respiratory failure, respiratory shock, septic shock, cytokine storm, organ failure, hypoxemia, acute cardiac injury, acute lung injury, inflammation of cells in the large away and parenchyma, intra-alveolar edema, lymphopenia, perivascular cuffing, RNAemia, thickening of the interstitial membrane, hemorrhage or clotting, and a neurological condition.

19. The method of claim 18, wherein the subject is exhibiting hemorrhage or clotting.

20. The method of claim 19, wherein the subject is exhibiting one or more of venous thromboembolism, arterial thrombosis, microvascular thrombosis, and bleeding.

21. The method of claim 18, wherein the subject is exhibiting a neurological condition.

22. The method of claim 21, wherein the subject is exhibiting one or more of a smell and taste disorder, encephalopathy, stroke, venous sinus thrombosis, a neuromuscular disease, meningoencephalitis, encephalomyelitis, multisystem inflammatory syndrome, myoclonus, and posterior reversible encephalopathy.

23. The method of any one of claims 1-10, wherein the coronavirus is SARS-CoV-2, and the subject is exhibiting post-COVID syndrome.

24. The method of any one of claims 1-23, wherein the risk of death of the subject is reduced.

25. The method of any one of claims 1-24, wherein the risk or duration of hospitalization of the subject is reduced.

26. The method of claim 25, wherein the subject is hospitalized, and the duration of hospitalization of the subject is reduced.

27. The method of any one of claims 1-26, wherein the risk of developing one or more symptoms selected from fatigue, muscle or body aches, congestion, nausea, vomiting, diarrhea, rhinorrhea, coughing, sneezing, lung ground-glass opacity, hemoptysis, nasal congestion, fever, sputum production, sore throat, chills, postnasal discharge, tonsillar hypertrophy, shortness of breath, headache, pneumonia, bronchiolitis, acute respiratory distress syndrome, respiratory failure, respiratory shock, septic shock, cytokine storm, organ failure, hypoxemia, acute cardiac injury, acute lung injury, inflammation of cells in the large away and parenchyma, intra-alveolar edema, lymphopenia, perivascular cuffing, RNAemia, and thickening of the interstitial membrane, hemorrhage or clotting, and a neurological condition is reduced.

28. The method of claim 27, wherein the risk of developing hemorrhage or clotting is reduced.

29. The method of claim 28, wherein the risk of developing one or more of venous thromboembolism, arterial thrombosis, microvascular thrombosis, and bleeding is reduced.

30. The method of claim 27, wherein the risk of developing a neurological condition is reduced.

31. The method of claim 29, wherein the risk of developing one or more of a smell and taste disorder, encephalopathy, stroke, venous sinus thrombosis, a neuromuscular disease, meningoencephalitis, encephalomyelitis, multisystem inflammatory syndrome, myoclonus, and posterior reversible encephalopathy is reduced.

32. The method of any one of claims 1-10, wherein the coronavirus is SARS-CoV-2, and the risk of developing post-COVID syndrome is reduced.

33. The method of any one of claims 1-32, wherein the risk or duration of need for non-invasive mechanical ventilation is reduced.

34. The method of claim 33, wherein the subject is being subjected to non-invasive mechanical ventilation intubation, and the duration of need for non-invasive mechanical ventilation of the subject is reduced.

35. The method of claim 1-34, wherein the risk or duration of need for extracorporeal membrane oxygenation is reduced.

36. The method of any one of claims 1-35, wherein the risk or duration of need for invasive mechanical ventilation is reduced.

37. The method of claim 36, wherein the risk or duration of need for respiratory intubation of the subject is reduced.

38. The method of claim 36, wherein the risk or duration of need for high-flow oxygen therapy is reduced.

39. The method of any one of claims 34-36, wherein the subject is being subjected to invasive mechanical ventilation intubation, and the duration of need for invasive mechanical ventilation of the subjection is reduced.

40. The method of any one of claims 1-39, wherein the FGF is administered parenterally.

41. The method of claim 40, wherein the FGF is administered intravenously, intramuscularly, or subcutaneously.

42. The method of any one of claims 1-39, wherein the therapeutic agent is intranasally administered.

43. The method of any one of claims 1-39, wherein the therapeutic agent is administered via inhalation.

44. The method of any one of claims 1-39, wherein the subject is being subjected to invasive-mechanical ventilation, and the therapeutic agent is administered via an endotracheal tube.

45. The method of any one of claims 1-39, wherein said administering of the FGF comprises administering a vector comprising a recombinant nucleic acid encoding the FGF.

46. The method of any one of claims 1-39, wherein said administering of the FGF comprises administering an mRNA encoding the FGF.

47. The method of any one of claims 1-46, wherein the subject has at least one pre-existing condition that increases the risk of one or more of pneumonia, acute respiratory distress syndrome, respiratory failure, septic shock, organ failure, cytokine storm, encephalopathy, stroke, hemorrhage, and death.

48. The method of claim 47, wherein the at least one pre-existing condition is selected from cardiovascular disease, chronic respiratory disease, diabetes, hypertension, immune deficiency, and obesity.

49. The method of any one of claims 1-48, wherein the FGF is administered in combination with one or more second therapeutic agents.

50. The method of claim 49, wherein the one or more second therapeutic agent comprises an antiviral agent.

51. The method of claim 50, wherein the antiviral agent is remdesivir.

52. The method any one of claims 1-51, wherein the second therapeutic agent comprises an anti-inflammatory agent.

53. The method of claim 52, wherein the anti-inflammatory agent is a steroid.

54. The method of claim 52, wherein the anti-inflammatory agent is a non-steroidal anti-inflammatory drug.

55. The method of any one of claims 1-54, wherein the second therapeutic agent comprises a glycosaminoglycan.

56. The method of claim 55, wherein the glycosaminoglycan is a heparin, a heparinoid, a fractionated low molecular weight heparin, or a heparin salt.

57. The method of claim 55 or 56, wherein the FGF is FGF-2, and the glycosaminoglycan is provided at a non-anticoagulant dose.

58. An FGF for use in the method of any one of claims 1-57.

59. Use of an FGF in the manufacture of a medicament for use in the method of any one of claims 1-57.
